SEQUENCE LISTING (1 sequence)

SEQ ID NO: 1 - protein, 380 aa, Homo sapiens
  ref <301>: AUTHORS: Haffner, C. Jarchau, T. Reinhard, M. Hoppe, J. Lohmann, S.M. Walter, U.
  ref <302>: TITLE: Molecular cloning, structural analysis and functional expression of the proline-rich focal adhesion and microfilament- associated protein VASP
  ref <303>: JOURNAL: EMBO J.
  ref <304>: VOLUME: 1995
  ref <305>: ISSUE: 14(1)
  ref <306>: PAGES: 19-27
  ref <313>: RELEVANT RESIDUES: 1 - 380
MSETVICSSRATVMLYDDGNKRWLPAGTGPQAFSRVQIYHNPTANSFRVVGRKMQPDQQVVINCAIVRGVKYNQATPNFHQWRDARQVWGLNFGSKEDAAQFAAGMASALEALEGGGPPPPPALPTWSVPNGPSPEEVEQQKRQQPGPSEHIERRVSNAGGPPAPPAGGPPPPPGPPPPPGPPPPPGLPPSGVPAAAHGAGGGPPPAPPLPAAQGPGGGGAGAPGLAAAIAGAKLRKVSKQEEASGGPTAPKAESGRSGGGGLMEEMNAMLARRRKATQVGEKTPKDESANQEEPEARVPAQSESVRRPWEKNSTTLPRMKSSSSVTTSETQPCTPSSSDYSDLQRVKQELLEEVKKELQKVKEEIIEAFVQELRKRGSP

(19) United States
(12) Patent Application Publication
(10) Pub. No.: US 2003/0166005 A1
(43) Pub. Date: Sep. 4, 2003

(54) Test system for determining the activity of cyclo-nucleotide-dependent protein kinases and vasp phosphatases

(76) Inventors: Peter Druckes, Freiburg (DE); Thomas Jarchau, Wurzburg (DE); Ulrich Walter, Veitschochheim (DE); Benjamin Bader, Berlin (DE)

Correspondence Address: FINNEGAN, HENDERSON, FARABOW, GARRETT & DUNNER, LLP, 1300 I STREET, NW, WASHINGTON, DC, 20005, US

(21) Appl. No.: 10/311,093

(22) PCT Filed: Jun. 12, 2001
(86) PCT No.: PCT/EP01/06621

(30) Foreign Application Priority Data
Jun. 14, 2000 (DE) 10029210.0

(51) Int. Cl. G01N 33/53; C12M 1/34

(52) U.S. Cl. 435/7.1; 435/287.2; 435/7.5

(57) ABSTRACT

The invention relates to an HTS-appropriate test system for detecting the activity of cyclo-nucleotide-dependent protein kinases (cNPK) containing: a) at least one test compound; b) at least one appropriate cNPK substrate; c) at least one composition, which is to be incubated and which contains cNPK and ATP, optionally, phosphorylation reaction stoppers, and; d) an appropriate detection system for quantifying the phosphorylation of the cNPK substrate. The invention also relates to an HTS-appropriate test system for detecting the activity of VASP phosphatases containing: e) at least one test compound; f) at least one appropriate VASP phosphatase substrate; g) at least one composition, which is to be incubated and which contains VASP phosphatase, and; h) an appropriate detection system for quantifying the dephosphorylation of the VASP phosphatase substrate. The described test systems can be used for locating compounds, which modulate the activity of a cNPK or of a VASP phosphatase, from chemical or natural substance libraries.

The present invention relates to a test system for detecting the activity of cyclonucleotide-dependent protein kinases or of phosphatases which dephosphorylate the vasodilator-stimulated phosphoprotein (“VASP”), to methods for preparing it and to its use in (high-throughput) screening methods.

Cyclonucleotide-dependent protein kinases (“cNPKs”) their substrates (“cNPKSs”), and also the corresponding protein phosphatases, are part of important physiologically, pathophysiologically and pharmacologically relevant cellular signal pathways (cf. references 1, 2 and 3). The cNPKs include cAMP-dependent protein kinases (“cAKs”) and cGMP-dependent protein kinases (“cGKs”) . Aside from other cGMP effector systems, such as cGMP-regulated phosphodiesterases and ion channels, cGK is, in particular, an important mediator of cGMP-mediated signal transmission.

The signals which lead to an increase in the intra-cellular level of cGMP are differentiated into those which bring this about by means of membrane-located guanylyl cyclases (GC-A, GC-B and GC-C), as, for example, by natriuretic peptides (abbreviated to: ANP, BNP and CNP) and enterotoxin/guanylin, and those which activate the soluble guanylyl cyclase (GC-S) . One of the most important agonists of the soluble guanylyl cyclase is the nitrogen monoxide (NO) which is formed by the NO synthases (NOS I-III). The established effects which are brought about by an increase of cGMP include, inter alia, the relaxation of smooth muscle cells (SMCs) and inhibition of blood platelet activation. These cGMP effects are mediated by (type 1) cGK (cf. references 1-3). It has been possible to confirm this in appropriate mouse models suffering from cGK-I deficiency (cf. references 4 and 5).

cGKs are known to be homodimers, of which a soluble form (76 kDa monomer; cGK I) and a membrane-located form (86 kDa monomer; cGK II) can be distinguished. The anchoring of cGK II to the membrane is mediated by way of N-terminal myristoylation (cf. references 1-3). Two isoforms of cGK I exist, i.e. cGK Iα and cGK Iβ. Interestingly, cGK I, which is universally described as the soluble form, is predominantly present in blood platelets in particulate form and partially present in particulate form in smooth muscle cells. A particularly high expression of cGK I is found in human blood platelets and in smooth muscle cells. Activation of cGK-I in these cells leads to the effects which have already been described above, such as a lowering of the intracellular level of Ca(2+), inhibition of blood platelets and contraction of smooth muscle cells.

In addition to others, the known substrates of cGK include, for example, the inositol 1,4,5-triphosphate receptor (IP3R) and VASP, and also p25, cystic fibrosis transmembrane regulator (CFTR) and 6-pyruvoyltetra-hydropterin synthase (PTPS).

Disturbances of the ANF/NO/cGMP signal pathway are known for the different forms of atherosclerosis and hypertension and what is termed “endothelial dysfunction”, i.e. a disturbance of endothelium-mediated vasodilatation, is an early sign of vascular diseases associated with atherosclerosis, hypertension and diabetes (cf. references 6 and 7). Current findings provide evidence that the activation and/or expression of soluble guanylyl cyclase, in particular, is pathologically decreased in these vascular diseases (cf. references 8-10). It follows conclusively from this that compounds/substances which are able to replace the NO/cGMP signal pathway are of very great therapeutic importance. This is verified by the success of NO donors, such as nitroprusside, nitroglycerin, molsidomine and, since very recently, by NO-independent sGC activators, such as YC-1 and derivatives. Despite these tested agents, there is still a great need for additional, novel substances since unsolved problems exist (development of tolerance, toxic side-effects, etc.). It would therefore be desirable to directly activate cGKs, preferably cGK-I, since this latter is still expressed in pathologically altered blood vessel segments (cf. references 10-11). The fact that not all the effects brought about by NO and/or cGMP are mediated by cGK holds the potential that influence can be exerted specifically on only some of the otherwise broad effects of this signal pathway and, as a result, some undesirable side-effects will probably not appear. So far, it is only cell membrane-permeable cGMP analogs (e.g. 8-Br-cGMP and 8-pCPT-cGMP) which have been described as being activators of cGK whereas certain cGMP analogs (Rp-8Br-PET-cGMPS and Rp-8-pCPT-cGMPS) and isoquinolinesulfonamide derivatives (KT 5823, H8 and H89) inhibit cGK (cf. reference 12).

There has so far not been any high-throughput screening approach for systematically searching for immediate cGK activators and/or inhibitors or for immediate activators and/or inhibitors of the VASP phosphatase. Combinatorial peptide libraries are used, in a laborious manner, to search for peptidergic cNPK substrates and inhibitors (cf. reference 13).

In the prior art, cNPK assays are customarily carried out by incorporating radioactive phosphorus isotopes (³²P or ³³P) into peptide substrates and subsequently separating these reaction products from the starting compounds, for example by immobilizing them on a carrier substance (reference 16), or by separating the reaction products by means of gel electrophoresis, and carrying out a radiochemical or an immunochemical detection following protein transfer to membranes when product-specific antibodies are available (as, for example, in a Western blot, WO 99/24,473). In the latter case, quantification of the formed product concentrations is particularly complicated matter, from the technical point of view, and only possible, in a manner which is not particularly reliable, as a semiquantitative determination relative to a chosen standard, and consequently not possible to be carried out for the purpose of precisely determining enzyme activities. In this connection, the acquisition and disposal of radioactive isotopes in the quantities which are required for this purpose, something which constitutes a burden from the point of view of costs and safety regulations, can be disadvantageous. In addition, measuring radioactive samples with the requisite precision requires a relatively large amount of time. In particular, gels are expensive as far as materials are concerned and are very time-consuming to process.

Since the methods which are known in the prior art involve separation and washing steps, these methods are unsuitable with regard to using automated systems to analyze a large number of test compounds.

A particular advantage of the present invention which has emerged is that the test system (synonymous: assay or diagnostic agent) which is described is suitable for being able to analyze, in a simple manner, a large number of (test) compounds, preferably from chemical or natural substance libraries, for their effect on the activity of a cNPK [so-called high-throughput screening (abbreviated to HTS)].

A further, particular advantage of the present invention which has emerged is that it is possible to use this test system, in a simple and effective manner, to analyze a large number of (test) compounds from chemical or natural substance libraries for their effect on the activity of a VASP phosphatase [so-called high-throughput screening (abbreviated to HTS)].

It has now been found that it is possible to detect the activity of a cNPK, or of a protein phosphatase, in the described test system even without separating the products and without using radioactive phosphorus isotopes (³²p or ³³P), with this method of detection overcoming the above-described disadvantages in the prior art and consequently being suitable for HTS, for example in an automated unit.

One part of the subject matter of the present invention is therefore an HTS-suitable test system for detecting the activity of a cNPK, comprising

a) at least one test compound,

b) at least one suitable cNPK substrate (cNPKS),

c) at least one composition which is to be incubated and which contains cNPK and ATP, and

d) a suitable detection system for quantitatively determining the quantity of the phosphorylated cNPK substrate.

Another part of the subject matter of the present invention is therefore an HTS-suitable test system for detecting the activity of the VASP phosphatase, comprising

e) at least one test compound,

f) at least one suitable VASP phosphatase substrate,

g) at least one composition which is to be incubated and which contains VASP phosphatase, and

h) a suitable detection system for quantitatively determining the quantity of the dephosphorylated VASP phosphatase substrate.

Both the abovementioned parts of the subject matter are described below as being test system according to the invention.

As a gel-free test system, the test system according to the invention makes it possible to carry out a large number of individual quantitative determinations and, by means of dispensing with gel-electrophoretic separations steps, fulfills particular requirements with regard to speed, sample size and consumption of materials. Furthermore, the test system ensures reproducibility, robustness, solvent tolerance and automation.

The test system according to the invention makes it possible to search for one or more, identical or different test compounds.

The cNPK used is preferably a cGK or cAK or a functional variant which possesses the activity of cNPK. Such protein kinases can be used in purified form or as a crude extract, in particular as a constituent in protein mixtures derived from homogenates of biological origin, preferably of human origin.

The VASP phosphatase used can likewise be a functional variant which possesses the activity of a VASP phosphatase. VASP phosphatases can be used in purified form or as a crude extract, in particular as a constituent in protein mixtures derived from homogenates of biological origin, preferably of human origin.

VASP, as depicted in SEQ ID No. 1 (described in C. Haffner et al. in EMBO J., 14(1), 19-27 (1995)), containing preferably operable phosphorylation sites, namely serine-157, serine-239 and threonine-278, is very particularly preferred for use as a suitable cNPK substrate in accordance with feature (b). cNPKs can in principle phosphorylate all three residues, however, the cAMP-dependent protein kinase prefers serine 157 as the main phosphorylation site, while the cGMP-dependent protein kinase prefers serine-139. Threonine-278 is phosphorylated with comparable specificities by both kinases.

Phosphorylated VASP, corresponding to SEQ ID No. 1, which is phosphorylated at the preferably operable phosphorylation sites, specifically serine-157, serine-239 and threonine-278, is very particularly preferred for use as a suitable VASP phosphatase substrate in accordance with feature (b).

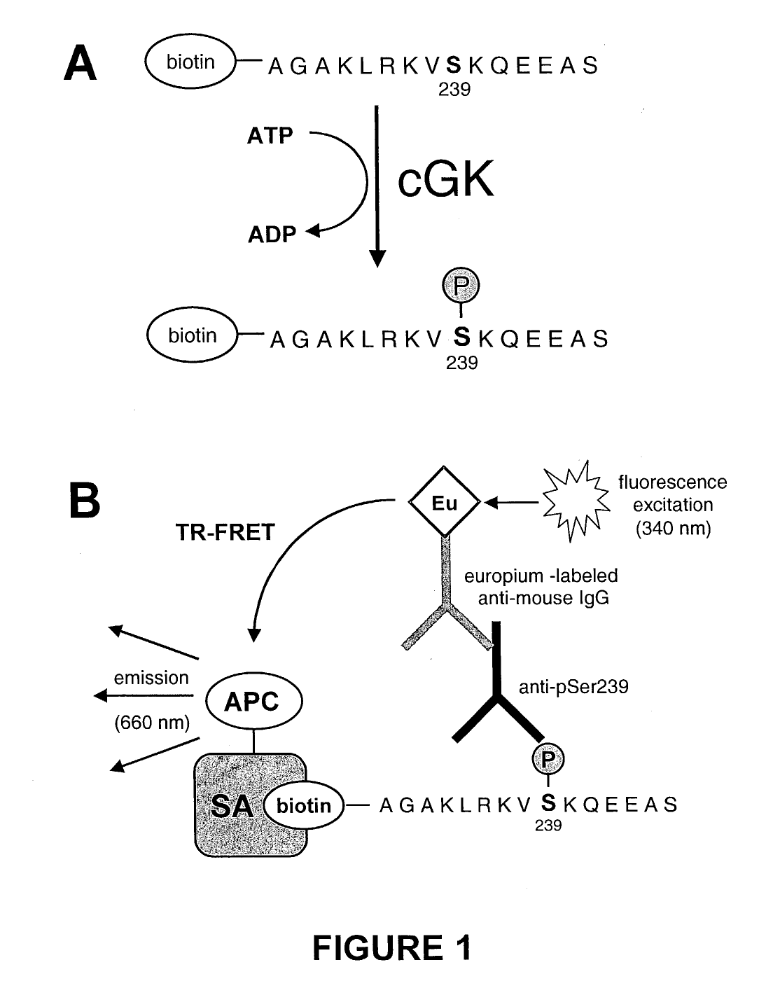

Within the context of the test system according to the invention, it is likewise possible to use, as functional variants of a cNPK substrate or of a VASP phosphatase substrate, those peptide or polypeptide variants, or peptoids, which preferably contain VASP amino acid sequences which are present in the environment of said phosphorylation sites or dephosphorylation sites, with particular preference being given, but not conclusively, to the pentamers containing amino acids 155-159 and/or 237-241 and/or 276-280 as depicted in SEQ ID No. 1, or preference being given to the decamers containing amino acids 152-161 and/or 234-243 and/or 273-282 from SEQ ID No. 1, or other suitable constituent sequences (e.g., cf. in FIG. 1).

These peptides, which are phosphorylated in accordance with the invention, can be particularly advantageously detected with suitable antibodies within the meaning of features (d) and (h), respectively, preferably monoclonal antibodies.

According to the invention, very particular preference is given to the monoclonal antibody 16C2, which is selected and obtainable from the hybridoma cell line DSM ACC2330 (WO 99/24473). This antibody, or a functional variant thereof, specifically detects a phosphorylation event; very particularly preferably it recognizes an epitope which contains the phosphorylated serine-239 of the VASP sequence. A functional variant relates, in particular, to those antibodies having a congruence of preferably 90-99% or 70-90% with the amino acid sequence of 16C2 with the homologous function of quantitatively determining a reaction product within the meaning of feature (d) or (h), respectively.

While the test system according to the invention can be operated heterogeneously (e.g. after immobilizing the reaction products) it is preferably operated homogeneously. Depending on the mode of operation, the immunological detection within the meaning of features (d) and (h), respectively, can be effected using methods which are known to the skilled person, such as a coupled enzyme reaction (alkaline phosphatase or luciferase) or general sandwich technologies (for example ELISA) and also fluorimetric methods (fluorescence, time-resolved fluorescence and fluorescence resonant energy transfer (FRET)).

Preference is given to antibodies which are radioactively labeled in a readily detectable manner, while particular preference is given to antibodies which are not radioactively labeled, in particular antibodies which are fluorescence-labeled. Lanthanide chelates are particularly suitable. Furthermore, it can particularly advantageously be reverted to the time-resolved fluorescence resonance energy transfer technique (FIG. 1). Such TR-FRET systems are described in WO 97/29373 and WO 98/15830 and can be obtained commercially through Wallac Oy (Turku, Finland) . Cf. “Miniaturization of a Lance™-assay” and “Use of Generic Reagent in Lance™”, published by Wallac Oy, Turku, Finland, as well.

Preferably, however, the test system according to the invention can be automated as a homogeneous HTS and is therefore suitable for 96-, 384- and 1536-well plates and more.

Further supplementary agents, such as buffer solutions, stabilizers and/or energy equivalents, in particular ATP, are preferably used for carrying out the investigations.

The present invention also relates to a method for preparing a test system in which at least one compound to be investigated and at least one composition, containing cNPK, ATP and cNPK substrate, where appropriate a phosphorylation reaction stopper, at least one suitable detection system, such as an antibody and an immunological detection means, and, where appropriate, further supplementary agents, are combined.

The present invention also relates to a method for preparing a test system in which at least one compound to be investigated, at least one composition containing VASP phosphatase and at least one detection system which is suitable for quantifying the dephosphorylation of a VASP phosphatase substrate, are combined.

Preferred embodiments of the individual components have already been described in detail above.

Another part of the subject matter of the present invention is an HTS-suitable method for finding one or more active compounds which modulate the activity of the cNPKs, comprising the steps of:

i) bringing the compound to be investigated, or a multiplicity of compounds to be investigated, into contact with cNPK, ATP and a suitable cNPK substrate, and

j) quantifying the phosphorylated CNPK substrate using a suitable detection system, optionally after stopping the phosphorylation reaction with a suitable phosphorylation reaction stopper.

Another part of the subject matter of the present invention is an HTS-suitable method for finding one or more active compounds which modulate the activity of the VASP phosphatase, comprising the steps of:

k) bringing the compound to be investigated, or a multiplicity of compounds to be investigated, into contact with VASP phosphatase and a suitable VASP phosphatase substrate, and

l) quantifying the dephosphorylated VASP phosphatase substrate using a suitable detection system, optionally after stopping the dephosphorylation reaction with a suitable dephosphorylation reaction stopper.

In a preferred embodiment, the method for finding a chemical compound, as described above, is carried out in a microtiter plate. In this connection, the microtiter plate can contain differing numbers of wells. For example, the plate can contain 96, 384, 768, 1536, 3072, or more, wells. Individual components of the method according to the invention which are preferred have already been described in detail above.

In this connection, the active (test) compound can be a pharmaceutically active compound, specifically having the function of a modulator, such as a (hyper)activator/or (total) inhibitor, of the activity of a cNPK, or be a natural product in the widest possible sense, in particular a crude extract or a component which is contained therein. The substance to be investigated is generally a naturally occurring, a naturally occurring and chemically modified and/or synthetic substance. In particular, it is possible to use the methods according to the invention to screen so-called combinatorial substance libraries particularly simply and rapidly.

Such a compound is very particularly preferably suitable which, while circumventing the NO/cGMP signal pathway, modulates a cNPK directly—activates or inhibits; optionally, selectively modulates—activates or inhibits,.

For this reason, the invention also relates to a method in which the pharmaceutically active compound modulates a cNPK signal pathway.

It has already been pointed out in the introduction to the description that various diseases can be attributed to a disturbance of the cyclonucleotide-dependent protein kinases (cNPKs) . For this reason, the present invention is also suitable for diagnosing a disease.

Another part of the subject matter of the present invention is therefore a method for diagnosing by directly, and in a gel electrophoresis-independent manner, determining the cNPK activity in samples, comprising the steps of:

m) incubating a sample in the presence of at least one composition containing a suitable cNPKS and, optionally ATP,

n) adding at least one detection system which is suitable for quantifying the phosphorylation of the cNPKS to the composition; and

o) determining the activity of the cNPK, in particular in blood extracts, cell extracts or tissue extracts or in samples which are in the form of permeabilized and/or intact cells.

Another part of the subject matter of the invention is a method for diagnosing by directly, and in a gel electrophoresis-independent manner, determining the VASP phosphatase activity in samples, comprising the steps of:

p) incubating a sample in the presence of at least one composition containing a suitable VASP phosphatase substrate,

q) adding at least one detection system which is suitable for quantifying the dephosphorylation of the VASP phosphatase substrate to the composition; and

r) determining the activity of the VASP phosphatase, in particular in blood extracts, cell extracts or tissue extracts or in samples which are in the form of permeabilized and/or intact cells.

In this connection, the diseases which are to be diagnosed are preferably angiocardiopathies and diseases which are associated with vascular damage, for example hypertension, thrombosis and the syndrome of endothelial dysfunction, for example in arteriosclerosis, diabetes, vasculites and diseases of hematopoietic cells, such as acute leukemias, myeloproliferative diseases and myelodysplasias (cf. reference 17).

The following Figures and Examples are intended to describe the invention in more detail without restricting it.

FIG. 1 shows a preferred embodiment of the test system according to the invention. The assay of cGK activity is based on observing the in-vitro phosphorylation of a biotinylated substrate peptide by activated cGK (compare FIG. 1A). The VASP substrate peptide consists of a part of the amino acid sequence of human VASP (vasodilator-stimulated phosphoprotein), which is a natural cGK substrate protein. Surprisingly, it was possible to detect the phosphorylated VASP substrate peptide using a detection system which is based on time-resolved fluorescence resonance energy transfer (FRET) (compare FIG. 1B). In this connection, main components, possessing the following chemical properties, were present in the detection system in a particular mixing ratio:

1. The anti-pSer239-VASP (mAb 16C2) monoclonal antibody, which, astonishingly, specifically recognized the phosphorylated VASP substrate peptide (and left unphosphorylated VASP substrate peptide unrecognized).

2. A europium-labeled anti-mouse IgG second antibody, which can specifically bind to the anti-pSer239-VASP (mAb 16C2).

3. Fluorescence-labeled streptavidin (SA-APC), which can bind the biotin group of the VASP substrate peptide.

When all three components, and the phosphorylated VASP substrate peptide, were mixed in a particular ratio, and when the light excitation (340 nm) of the europium-labeled antibody was suitable, there was found to be a fluorescence resonance energy transfer (FRET) to the second fluorescent component, i.e. SA-APC, whose light emission could be observed at 660 nm. This test system possessed the interesting properties seen in the following Figures and Examples.

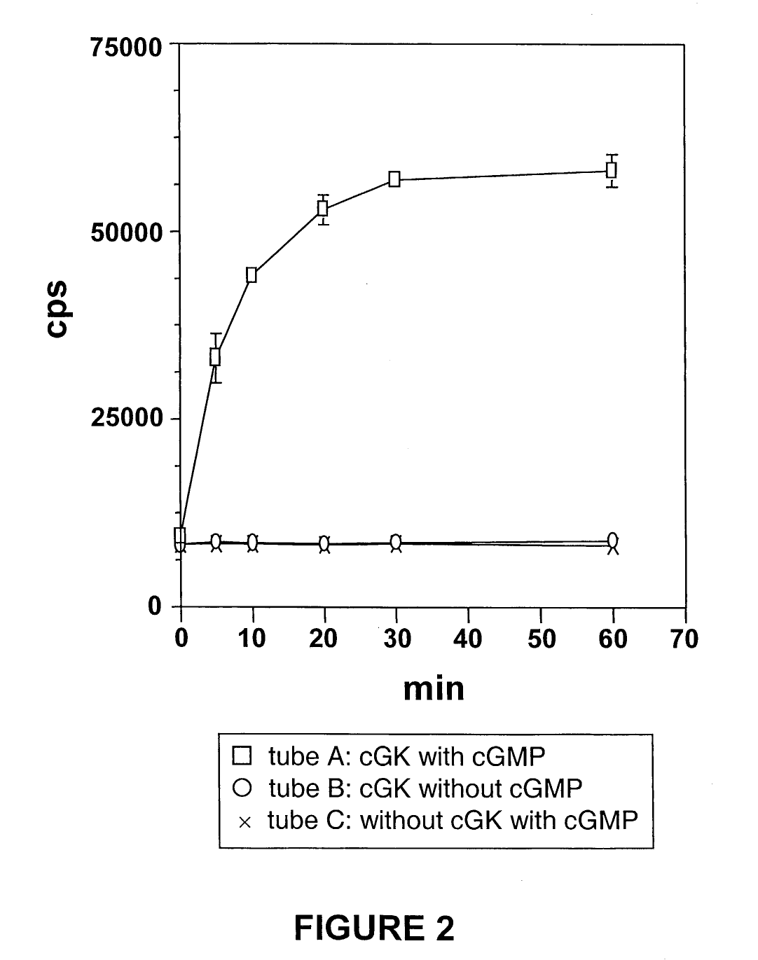

FIG. 2 shows the kinetics of the phosphorylation of the biotinylated substrate peptide by the cGK. The ordinate in FIG. 2 shows the FRET signal measured in cps. The VASP substrate peptide is only phosphorylated in the presence of the cGK and its activator cGMP (tube A). No change in signal was observed without cGMP and with cGK (tube B) and without cGK and with cGMP (tube C).

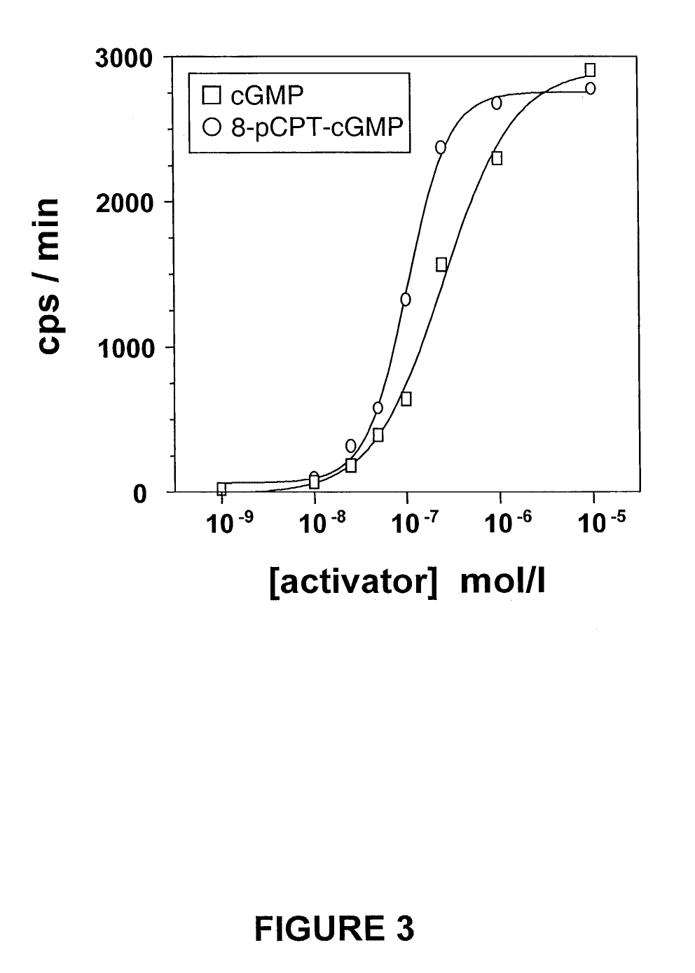

FIG. 3 shows the activation of the cGK with the natural activator cGMP and with a cGMP-analogous molecule (8-pCPT-cGMP), as measured using the described test system. The enzyme activity (in cps/min) is plotted against the activator concentration (in mol/l).

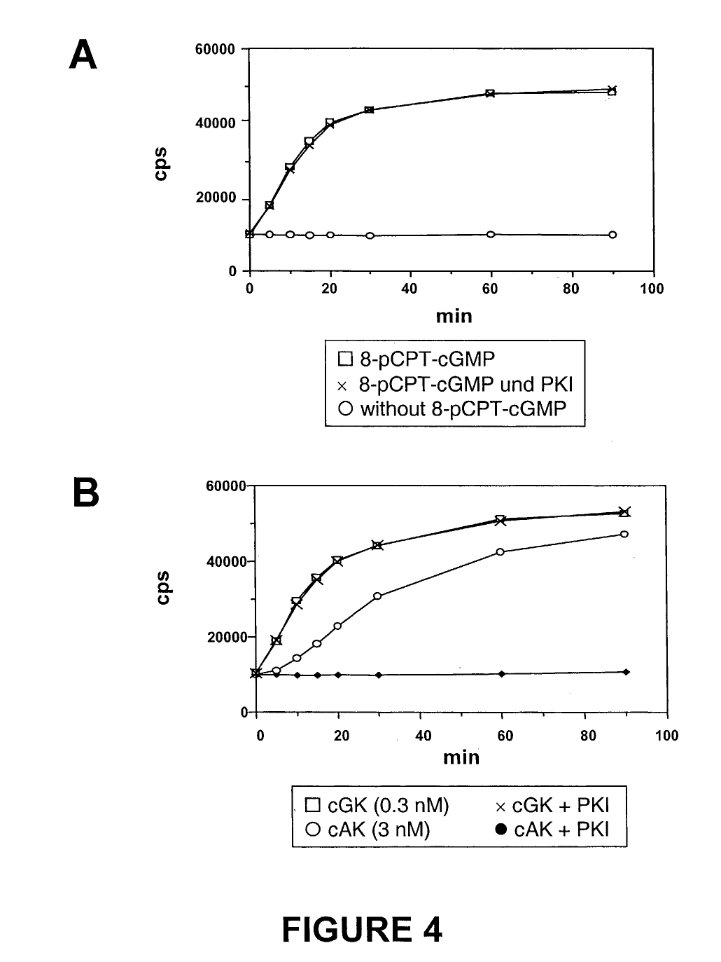

FIG. 4 shows the surprising finding that the test system according to the invention can be used to observe, and quantitatively measure, the enzyme activity of the cGK contained in thrombocyte extracts even in this complex biological mixture of protein. FIG. 4A shows no difference between the phosphorylation kinetics without (□) and with (×) the specific cAK inhibitor PKI. FIG. 4B depicts the influence of 100 nmol of PKI/l on the phosphorylation of the VASP substrate peptide by purified cGK (0.3 nmol/l) without (□) and with (×) PKI, and also the influence of PKI on purified cAK (catalytic subunit, 3 nmol/l) without (◯) and with (●) PKI.

### EXAMPLE 1

FRET Detection System and Use of Phosphopeptide to Titrate the Measurement Range

Instead of carrying out a kinase reaction for the purpose of establishing and optimizing the detection system, a synthetic phosphopeptide (phosphorylated version of the VASP substrate peptide) was used as the “reaction product” and was quantified using a detection mix. In order to ascertain the linear measurement range, different mixtures of unphosphorylated peptide and phosphopeptide were prepared. When so doing, the total concentration of peptide was kept constant at 500 nmol/l. 50 μl of these mixtures were added to the wells of a 96-well microtest plate and in each case treated with 200 μl of a detection mix which contained the following reagents diluted in PBS:

| | | |
|---|---|---|
| anti-pSer239-VASP (mAb 16C2) | 1 | nmol/l |
| anti-mouse-Eu (W 1024, PE Wallac) | 1 | nmo/l |
| Streptavidin-APC | 2 | μg/ml |
| Bovine serum albumin | 0.1 | g/l |

The time-resolved FRET signal, when exciting the europium fluorescence at 340 nm and measuring the emission of the APC fluorescence at 660 nm, was measured, after incubating at 20° C. for 1 hour, using a PerkinElmer Wallac fluorescence-measuring instrument (Victor²). The following Table shows a virtually linear increase between 0 and 100 nmol/l of phosphopeptide. At 160 nmol/l of phosphopeptide, the signal cannot be increased any further.

TABLE 1
Using phosphopeptide to titrate the
measurement range

| Concentration of | Quantity of | |
|---|---|---|
| phosphopeptide | substance in 100 μl | FRET signal |
| nmol/l | Pmol | Cps |
| 0 | 0 | 6218 |
| 10 | 1 | 7219 |
| 20 | 2 | 9872 |
| 40 | 4 | 15358 |
| 80 | 8 | 26720 |
| 160 | 16 | 30725 |
| 320 | 32 | 29923 |

The signal/background ratio, as defined by the quotient of the maximum signal and signal without phosphopeptide, was about 5. For detection in 384-well plates, an optimum signal/background ratio was obtained when 20 μl of a solution containing 500 nmol of VASP substrate peptide/l were mixed with 40 μl of a detection mix which contained the abovementioned concentrations of the detection reagent. This protocol was used in all the subsequent experiments.

### EXAMPLE 2

Quantifying the Enzyme Activity

The linearity of the measurement range when titrating with the aid of synthetically prepared phosphopeptide, as found in Example 1, Table 1, makes it possible to standardize and quantify the enzymatic kinase reactions on the basis of the test system according to the invention. The measured signal, expressed in cps, can be converted directly into molar concentration of phosphorylated peptide, thereby making it possible to convert the activity which is measured in the described test system into international enzyme units (1 unit=1 μmol/min).

Consequently, the activity of an enzyme reaction which is carried out in a volume of 100 μl and in which a signal of 9872 cps is measured within 2 minutes corresponds to an activity which transforms 2 pmol of substrate in 2 minutes, corresponding to an enzyme activity of 1×10⁻⁶ U.

### EXAMPLE 3

Kinase Reaction

High concentrations of VASP substrate peptide (50-100 μmol/l) and enzyme are required in the customary radioactive kinase assays (cf. reference 16). When the test system described in FIG. 1 was used, it was not possible to detect any kinase activity at high substrate peptide concentrations. It was only possible, for the first time, to measure enzyme kinetics by means of FRET detection as well by decreasing the substrate concentration to 1 μmol/l (cf. FIG. 2).

However, the use of high enzyme concentrations at a low substrate concentration led to an increase in the unstimulated basal activity of the kinase; that is, activity was measured even without the specific activator cGMP, with it not being possible to increase this activity, or it only being possible to increase it to a trivial extent, by adding cGMP. It was possible to overcome this problem by decreasing the enzyme concentration to 50 ng/ml and less (cf. reference 14). As a result, the test system according to the invention can be used to measure the authentic activity of the cGK enzyme.

The following components were mixed in three reaction tubes:

| | | |
|---|---|---|
| Tris/HCl, pH 7.4 | 20 | mmol/l |
| MgCl₂ | 10 | mmol/l |
| β-Mercaptoethanol | 5 | mmol/l |
| BSA | 0.01 | g/l |
| Biotinylated VASP substrate peptide | 1 | μmol/l |
| (biotin-VASP 231-245) | | |

Tube A additionally contained 5 μmol/l of cGMP and 25 ng/ml of purified gGK.

Tube B additionally contained 25 ng/ml of purified gGK.

Tube C additionally contained 5 μmol/l of cGMP.

After the tubes had been preincubated at 30° C. for 5 minutes, the kinase reaction was started with 50 μmol of ATP/l, with subsequent incubation being at 30° C. All the above-mentioned concentrations are to be understood as meaning final concentrations in a volume of 500 μl. At the given times, and for the purpose of terminating the reaction, 50 μl were removed from the tubes and mixed with 50 μl of a 30 mmol/l solution of EDTA. The phosphorylated substrate peptide was detected, as described in Example 1, in the wells of a 384-well microtest plate.

It can be seen from FIG. 2 that the VASP substrate peptide is only phosphorylated in the presence of cGK and its activator cGMP (tube A). Without cGMP and with cGK (tube B), as well as without cGK and with cGMP (tube C), no change in signal was observed.

### EXAMPLE 4

Suitability for HTS

In the form described, the test system according to the invention is suitable for high-throughput screening. Microtest plates in the 96-well, 384-well and even 1536-well format can be processed using commercially available pipetting robots (e.g. the Biomek Systems from Beckman or the Genesis Workstation from Tecan). The cGK assay could be implemented in the following way, on such a robot system in the 384-well format, for screening for activators of cGK. The following are pipetted in sequentially:

1. 20 μl of a solution containing buffer, cGK and substrate peptide

2. 5 μl of a test substance in DMSO

3. Reaction started with 5 μl of an ATP solution

4. Reaction stopped by simultaneously adding EDTA and the detection reagents.

Most substances which are tested in an HTS are preferably dissolved in DMSO. However, many enzymes or test systems are very sensitive to DMSO. In order to determine the influence of DMSO on the test system according to the invention, phosphorylation kinetics, as described in Example 2 (cf. FIG. 2), were carried out in the presence of 0, 5, 10, 15 and 20% DMSO. Table 2 summarizes the enzyme activities which were obtained after an incubation period of 10 min. The test system according to the invention turns out to be very robust in this connection. Adding up to 20 percent by volume of DMSO has no significant influence on the reaction.

TABLE 2
Influence of DMSO on the enzyme activity in
the test system according to the invention

| Percent by | Enzyme activity |
|---|---|
| volume of DMSO | in cps/min |
| 0 | 2248 |
| 5 | 2282 |
| 10 | 2253 |
| 15 | 2218 |
| 20 | 1988 |

### EXAMPLE 5

cGK Activation Curve

The test system according to the invention can be used to analyze the dose-dependent activation of cGK by the natural activator cGMP and a molecule which is analogous to cGMP (8-pCPT-cGMP). 8 kinase reactions (as described in Example 3, total volume 500 μl) were carried out, with these reactions containing the following components:

20 mmol/l of Tris/HCl, pH 7.4

10 mmol/l of MgCl₂

5 mmol/l of β-mercaptoethanol

0.01 g/l of BSA

1 μmol/l of biotinylated VASP substrate peptide (biotin-VASP 231-245)

25 ng/ml of cGK

50 μmol/l of ATP

various concentrations of cGMP or 8-pCPT-cGMP of 10⁻⁹-10⁻⁶ mol/l.

After 0, 5, 10, 20 and 60 minutes of incubation at 30° C., partial volumes of 50 μl were removed and mixed with 50 μl of 30 μmol/l of EDTA. The phosphorylated substrate peptide was then detected as described in Example 2. The respective initial rates, in cps/min, at various activator concentrations were calculated from the kinetics of the phosphorylation of the VASP substrate peptide. These rates are plotted against the activator concentration in FIG. 3.

The activation curves which were obtained using the test system according to the invention, and the activation constants for cGMP (0.26 μmol/l) and 8-pCPT-cGMP (0.10 μmol/l) which were calculated from them, are comparable with data which have been published for these substances and which were determined using conventional radioactive methods (cf. reference 14).

### EXAMPLE 6

Determining the cGK Activity in Tissue Extracts

The test system according to the invention is suitable for specifically measuring the activity of cGK in tissue extracts or thrombocyte lysates.

Thrombocytes were prepared using a known method (cf. reference 15). After a soluble thrombocyte extract had been prepared, it was tested without activator and with 8-pCPT-cGMP (0.5 μmol/l) as described in Example 3. In addition to 8-pCPT-cGMP, a third reaction tube also contained 100 nmol/l of PKI. This inhibitor specifically inactivates cAMP-dependent protein kinase (cAK), which is also present in thrombocytes. FIG. 4A shows no difference between the phosphorylation kinetics without and with PKI. FIG. 4B depicts the influence of 100 nmol of PKI/l on the phosphorylation of the VASP substrate peptide by purified cGK (0.3 nmol/l) and purified cAK (catalytic subunit, 3 nmol/l) . The concentration of PKI which is employed is sufficient to inhibit the cAK completely. The activity of the cGK is unaffected.

FIGS. 4A and 4B therefore together show that the test system according to the invention can measure the cGK activity in complex samples, such as thrombocyte lysates, very specifically. Any cross reactivity due to cAK can be ruled out. It was likewise possible to measure cGK activity in tissue homogenates prepared from rat organs.

Abbreviations Used in the Text and Figures:

8-pCPT=8-(4-chlorophenylthio)-

cAMP=cycloadenosine-3′, 5′-monophosphate

APC=allophycocyanin

ATP=adenosine-5′-triphosphate

BSA=bovine serum albumin

cAK=cAMP-dependent protein kinase

cGK=cGMP-dependent protein kinases

cGMP=cycloguanosine-3′, 5′-monophosphate

cNPKS=substrate for cyclonucleotide-dependent protein kinases

cNPK=cyclonucleotide-dependent protein kinases

DMSO=dimethyl sulfoxide

Eu=europium

HTS=high-throughput screening

LANCE=lanthanide chelate excitation technology

mAb=monoclonal antibody

PKI=protein kinase A-specific inhibitor

SA=streptavidin

TR-FRET=time-resolved fluorescence resonance energy transfer

VASP=vasodilator-stimulated phosphoprotein

Literature Cited in the Text:

1. Lohmann, S. M., Vaandrager, A. B., Smolenski, A., Walter, U. and DeJonge, H. R. (1997) Distinct and specific functions of cGMP-dependent protein kinases. TIBS 22, 307-312.

2. Pfeifer, A., Ruth, P., Dostmann, W., Sausbier, M., Klatt, P. and Hofmann, F., (1999) Structure and Function of cGMP-Dependent Protein Kinases, Rev. Physiol. Biochem. Pharmacol. 135, 105-149.

3. Eigenthaler M, Lohmann S M, Walter U and Pilz R B (1999) Signal transduction by cGMP-dependent protein kinases and their emerging roles in the regulation of cell adhesion and gene expression, Rev. Physiol. Biochem. Pharmacol. 135, 173-209.

4. Pfeiffer A, Klatt P, Massberg S, Ny L, Sausbier M, Hirneis C, Wang G X, Korth M, Aszodi A, Andersson K E, Krombach F, Mayerhofer A, Ruth P, Fässler R, and Hofmann F (1998) Defective smooth muscle regulation in cGMP kinase I-deficient mice, EMBO J 17, 3045-3051.

5. Massberg S, Sausbier M, Klatt P, Bauer M, Pfeifer A, Siess W, Fässler R, Ruth P, Krombach F, and Hofmann F (1999) Increased adhesion and aggregation of platelets lacking cyclic guanosine 3′-5′ monophosphate kinase I. J. Exp. Med. 189, 1255-1263.

6. Ross R (1999) Atherosclerosis—An Inflammatory Disease. N. Engl. J. Med. 340, 115-126.

7. Garbers D L, and Dubois, S K (1999) The Molecular Basis of Hypertension. Annu. Rev. Biochem. 68, 127-155.

8. Bauersachs J, Bouloumie A, Mulsch A, Wiemer G, Fleming I, and Busse R (1998) Vasodilator dysfunction in aged spontaneously hypertensive rat: changes in NO synthases III and soluble guanylyl cyclase expression, and in superoxide anion production. Cardiovascular Res. 37, 772-779.

9. Ruetten H, Zabel U, Linz W, Schmidt H H (1999) Down-regulation of soluble guanylyl cyclase in young and aging spontaneously hypertensive rats. Circ. Res. 85, 534-541.

10. Bauersachs J (1999) Endothelial dysfunction in chronic myocardial infarction despite increased vascular endothelial nitric oxide synthase and soluble guanylyl cyclase expression: role of enhanced superoxide production. Circulation 100, 292-298.

11. Mönks D, Lange V, Silber R E, Markert T, Walter U and Nehls V (1998) Expression of cGMP-dependent protein kinase and its substrate VASP in neointimal cells of the injured rat carotid artery. Eur. J. Clin. Invest. 28, 416-423.

12. Smolenski A, Burkhardt A M, Eigenthaler M, Butt E, Gambaryan S, Lohmann S M and Walter U (1998) Functional analysis of cGMP-dependent protein kinases I and II as mediators of NO/cGMP effects. Naunyn-Schmiedenberg's Arch. Pharmacol. 358, 134-139.

13. Dostmann W R G, Nickl C, Thiel S. Tsigelny I, Frank R and Tegge W J (1999) Delineation of selective cyclic GMP-dependent protein kinase Iα substrate and inhibitor peptides based on combinatorial peptide libraries on paper. Pharmcol. Ther. 82, 373-387.

14. Sekhar, K. R., Hatchett, R. J., Shabb, J. B., Wolfe, L., Francis, S. H., Wells, J. N., Jastorff, B., Butt, E., Chakinala, M. M., and Corbin, J. D. (1992). Relaxation of pig coronary arteries by new and potent cGMP analogs that selectively activate type I alpha, compared with type I beta, cGMP-dependent protein kinase. Mol. Pharmacol. 42: 103-8.

15. Eigenthaler, M., Nolte, C., Halbrugge, M., and Walter, U. (1992). Concentration and regulation of cyclic nucleotides, cyclic-nucleotide-dependent protein kinases and one of their major substrates in human platelets. Estimating the rate of cAMP-regulated and cGMP-regulated protein phosphorylation in intact cells. Eur. J. Biochem. 205: 471-81.

16. Roskoski, R., Jr. (1983). Assays of protein kinase. Methods Enzymol. 99: 3-6.

17. Eigenthaler, M., Ullrich, H., Geiger, J., Horstrup, K., Honig-Liedl, P., Wiebecke, D., and Walter, U. (1993). Defective nitrovasodilator-stimulated protein phosphorylation and calcium regulation in cGMP-dependent protein kinase-deficient human platelets of chronic myelocytic leukemia. J. Biol. Chem. 268: 13526-31.

1. An HTS-suitable test system for detecting the activity of the cyclonucleotide-dependent protein kinases (cNPKs), comprising
a) at least one test compound,
b) at least one suitable cNPK substrate (cNPKS),
c) at least one composition which is to be incubated and which contains cNPK and ATP, and
d) a suitable detection system for quantitatively determining the quantity of the phosphorylated cNPK substrate.

2. An HTS-suitable test system for detecting the activity of the VASP phosphatase, comprising
e) at least one test compound,
f) at least one suitable VASP phosphatase substrate,
g) at least one composition which is to be incubated and which contains VASP phosphatase, and
h) a suitable detection system for quantitatively determining the quantity of the dephosphorylated VASP phosphatase substrate.

3. A test system according to claim 1 or 2, which can be automated and is heterogeneous, with separation or immobilization of at least one of the reaction products or detection reagents.

4. A test system according to claim 1 or 2, which can be automated and is homogeneous.

5. A test system according to any of claims 1 to 4, which is nonradioactive.

6. A test system according to claim 1 or 2, characterized in that the cNPK substrate according to feature (b) or the VASP phosphatase substrate according to feature (f) is selected from SEQ ID No. 1 or a functional variant thereof.

7. A test system according to claim 6, characterized in that the cNPK substrate is a peptide, polypeptide or peptoid comprising the pentamers containing amino acids 155-159 and/or 237-241 and/or 276-280 from SEQ ID No. 1 or the decamers containing amino acids 152-161 and/or 234-243 and/or 273-282 from SEQ ID No. 1.

8. A test system according to claim 1, characterized in that the cNPK is preferably a cAMP kinase and/or cGMP kinase, or a functional variant thereof.

9. A test system according to claim 8, characterized in that the cNPKs are present in purified form, in the form of blood extracts, cell extracts or tissue extracts, or in the form of permeabilized and/or intact cells.

10. A test system according to any of claims 1 to 9, characterized in that the detection system for quantifying the phosphorylation of the cNPK substrate or the dephosphorylation of the VASP phosphatase substrate contains at least one antibody which is suitable for the phosphorylated product or for the dephosphorylated product.

11. A test system according to claim 10, characterized in that the antibody is formed by the hybridoma cell line DSM ACC2330, and is, in particular, the monoclonal antibody 16C2 or a functional variant thereof.

12. A test system according to claim 10 or 11, characterized in that the detection system comprises two antibodies, with the first antibody being unlabeled and the second antibody being labeled.

13. A test system according to claim 10 or 11, characterized in that the detection system comprises at least one labeled antibody and also a suitable system for detecting the label.

14. A test system according to claim 10 or 11, characterized in that the antibody contains a fluorophor.

15. A test system according to any of claims 1 to 14, characterized in that the detection system is based on fluorescence resonance energy transfer (FRET), with use being made of at least one labeled, preferably one fluorophor-labeled, first antibody or second antibody and an acceptor fluorophor or donor fluorophor which corresponds thereto, preferably on the cNPK substrate.

16. A test system according to claim 15, characterized in that the detection system is based on a time-resolved FRET system using lanthanide chelates, preferably europium chelates.

17. A test system according to any of claims 1 to 16, characterized in that buffer solutions, stabilizers and/or energy equivalents are employed as further supplementary agents.

18. A method for preparing a test system according to claim 1, characterized in that at least one compound to be investigated and at least one composition containing cNPK and ATP, and at least one detection system which is suitable for quantitatively determining the quantity of the phosphorylated cNPK substrate are combined.

19. A method for preparing a test system according to claim 2, characterized in that at least one compound to be investigated, at least one composition containing VASP phosphatase and at least one detection system which is suitable for quantitatively determining the quantity of the dephosphorylated VASP phosphatase substrate are combined.

20. An HTS-suitable method for finding one or more active compounds which modulate the activity of the cNPKs, comprising the steps of
i) bringing the compound to be investigated, or a multiplicity of compounds to be investigated, into contact with cNPK, ATP and a suitable cNPK substrate, and
j) quantifying the phosphorylated cNPK substrate using a suitable detection system.

21. An HTS-suitable method for finding one or more active compounds which modulate the activity of the VASP phosphatase, comprising the steps of:
k) bringing the compound to be investigated, or a multiplicity of compounds to be investigated, into contact with VASP phosphatase and a suitable VASP phosphatase substrate, and
l) quantifying the dephosphorylated VASP phosphatase substrate using a suitable detection system.

22. A method according to any of claims 20 to 21, characterized in that the compound to be investigated is selected from a naturally occurring, naturally occurring and chemically modified and/or synthetic compound.

23. A method according to claim 22, characterized in that the compound to be investigated is used in the form of a combinatorial substance library.

24. A method for diagnosing by directly, and in a gel electrophoresis-independent manner, determining the cNPK activity in samples, comprising the steps of:
m) incubating a sample in the presence of at least one composition containing a suitable cNPKS and ATP,
n) adding at least one detection system which is suitable for quantifying the phosphorylation of the cNPKS to the composition; and
o) determining the activity of the cNPK, in particular in blood extracts, cell extracts or tissue extracts or in samples which are in the form of permeabilized and/or intact cells.

25. A method for diagnosing by directly, and in a gel electrophoresis-independent manner, determining the VASP phosphatase activity in samples, comprising the steps of:
p) incubating a sample in the presence of at least one composition containing a suitable VASP phosphatase substrate,
q) adding at least one detection system which is suitable for quantifying the dephosphorylation of the VASP phosphatase substrate to the composition; and
r) determining the activity of the VASP phosphatase, in particular in blood extracts, cell extracts or tissue extracts or in samples which are in the form of permeabilized and/or intact cells.

26. A method according to claim 24 or 25, characterized in that the method is the diagnosis of a disease which is selected from the spectrum of angiocardiopathies and diseases which are associated with vascular damage, in particular hypertension, thrombosis and the endothelial dysfunction syndrome in arteriosclerosis, diabetes and vasculites, or diseases of hematopoietic cells, such as acute leukemia, myeloproliferative diseases or myelodysplasias.
